(19) United States
(12) Patent Application Publication
(10) Pub. No.: US 2023/0321154 A1
(43) Pub. Date: Oct. 12, 2023

(54) DENTAL AND PERIODONTAL APPLICATION OF FIBROBLASTS

(71) Applicant: FIGENE, LLC, Houston, TX (US)

(72) Inventors: Pete O'HEERON, Houston, TX (US); Thomas ICHIM, San Diego, CA (US)

(21) Appl. No.: 18/042,323

(22) PCT Filed: Aug. 27, 2021
(86) PCT No.: PCT/US2021/071296

§ 371 Date: Feb. 21, 2023

Related U.S. Application Data
(60) Provisional application No. 63/071,333, filed on Aug. 27, 2020.

(51) Int. Cl. A61K 35/33; A61K 9/00; A61C 13/08

(52) U.S. Cl. CPC A61K 35/33; A61K 9/0053; A61C 13/08

(57) ABSTRACT

Embodiments of the disclosure include means of treating a variety of periodontal and dental diseases using fibroblasts, modified fibroblasts, and derivatives thereof. In one embodiment, the disclosure encompasses the utilization of fibroblasts for treatment of gum disease. In another embodiment, disclosed are means of utilizing fibroblasts and modifications thereof, for preparing gum tissue for placement of an artificial tooth. In another embodiment, fibroblasts are utilized to increase efficacy of bone grafts. In other embodiments, fibroblasts are utilized to generate artificial teeth through utilization of various scaffolding and/or 3 dimensional printing means.

This application claims priority to U.S. Provisional Patent Application Ser. No. 63/071,333, filed Aug. 27, 2020, which is incorporated by reference herein in its entirety.

### TECHNICAL FIELD

Embodiments of the disclosure concern at least the fields of cell biology, molecular biology, biochemistry, cell therapy, dentistry, orthodontia, and medicine.

### BACKGROUND

It is known that periodontitis is a disease associated with inflammatory responses caused mainly by the periodontal plaque bacteria, although the host immune responses also play an important role. With the advent of systemic antibiotic therapy, solutions have included intrapocket devices for the treatment of periodontitis for more physiologically acceptable and commercially feasible drug delivery systems as an adjunct to the conventional surgical and nonsurgical treatments for periodontal infections.

The prevalence of periodontal attachment loss increases with age; 50 percent of 18- to 19-year old subjects; about 80 percent of 35- to 39-year old subjects; 87 percent for 45 to 49 years; and over 90 percent for those 60 years and older demonstrate symptoms. Most cases can be successfully treated by nonsurgical periodontal therapy (Phase I and Phase III). The vast majority of periodontal treatment needs should be addressed early by treating gingivitis and early periodontitis, preventing disease progression, and maintaining periodontal health following active periodontal therapy. Therapeutic scaling and root planing is performed to treat established periodontal disease at various levels found throughout the individual's dentition, and treatment decisions should be site specific for each tooth.

Studies have reported that Porphyromonas gingivalis is a Gram-negative oral anaerobe that is involved in the pathogenesis of periodontitis and is a member of more than 500 bacterial species that live in the oral cavity. This anaerobic bacterium is a natural member of the oral microbiome, yet it can become highly destructive (termed ‘pathobiont’) and proliferate to high cell numbers in periodontal lesions: this is attributed to its arsenal of specialized virulence factors. This bacterium, along with Treponema denticola and Tannerella forsythia, constitute the “red complex,” a prototype polybacterial pathogenic consortium in periodontitis. One of the mechanisms by which these bacterium cause pathology is secretion of proteases, which degrade gum and surrounding tissue. Additionally, stimulation of inflammatory processes also has been implicated in gum disease initiation and progression. Interestingly, these pathogens have also been suspected as having potential to initiate/accelerate other conditions such as arthritis, transplant rejection, atherosclerosis, and Alzheimer's Disease.

Currently there are no means of restoring gum tissue that has been lost because of infection. Additionally, current bone grafting means include the use of decellularized bone, as well as cytokines such as bone morphogenic protein (BMP)-2. Both of these approaches require significant timing for engraftment, as well as possess the risk of infection. The current disclosure provides means of protecting against gingival tissue loss through administration of fibroblasts and/or modified fibroblasts and/or modified fibroblast derivatives and/or fibroblasts associated with a device or scaffold.

### BRIEF SUMMARY

The present disclosure is directed to systems, methods, and compositions that are directed to the use of fibroblasts to treat or prevent a variety of oral medical conditions, including periodontal and dental diseases or medical conditions. In particular embodiments, a population of fibroblasts is utilized to treat at least gum disease. In specific embodiments, a population of fibroblasts and/or modifications thereof are used for preparing gum tissue for the placement of implants, such as for one or more artificial teeth. In some embodiments, a population of fibroblasts is used to increase efficacy of bone grafts in an oral location, including a dental environment.

In some embodiments, fibroblasts and/or media conditioned therefrom are provided to an individual in need thereof. In specific embodiments, the individual has a medical condition requiring replenishing of cells or tissues following onset of the medical condition, including at a localized site in some cases. In particular embodiments, an effective amount of the fibroblasts and/or conditioned medium therefrom and/or fibroblast-derived products is provided to an individual. The individual may have an oral medical condition for which fibroblasts and/or conditioned medium therefrom is therapeutic, allowing improvement of at least one symptom, for example. In specific embodiments, the oral medical condition is tooth loss, tooth decay, tooth impaction, gum disease, periodontitis, abscess, mouth ulcer, inflammation, leukoplakia, halitosis, infection, microbiome dysbiosis, bacterial microfilm, oral bacterial infections, oral viral infections, oral fungal infections, or combination thereof.

In particular embodiments, selection of fibroblasts for any one or more oral medical conditions, including dental applications, is performed from a population of fibroblasts that are extracted from a tissue, including skin, placenta, adipose, bone marrow, peripheral blood, omentum, and Wharton's jelly. In some embodiments, the fibroblasts are obtained from a punch biopsy and cultured under particular conditions, such as in a hypoxic environment, during tissue dissociation to yield a cellular population.

Embodiments of the disclosure include a method of enhancing a therapeutic activity of a plurality of fibroblasts for an oral indication, including a dental indication, and also including use thereof. In specific embodiments, the fibroblasts used in any method encompassed by the disclosure have regenerative activity, and in at least some cases the regenerative activity is present or enhanced because the fibroblasts have been modified and/or exposed to one or more particular conditions. In some embodiments, prior and/or during use the fibroblasts have been exposed to an effective amount of one or more agents and/or one or more conditions, such as stimulation of native heme oxygenase (HO)-1 expression in the fibroblasts, exposure of the fibroblasts to exogenously provided HO-1, and/or expression of HO-1 from an exogenously provided vector in the fibroblasts. The fibroblasts may have an increase in secretion of one or more cytokines and/or one or more growth factors; the fibroblasts may have an increase in anti-apoptotic activity; and/or the immunogenicity of the fibroblasts is modulated, such as reduced or increased, and such characteristics may be present because of modification of the fibroblasts and/or exposure to one or more certain agents and/or conditions. In particular embodiments, the one or more agents and/or conditions comprises hypoxia, carbon monoxide, or a combination thereof. The carbon monoxide may be provided to the fibroblasts prior to and/or subsequent to the hypoxia. The carbon monoxide may be provided to the fibroblasts at the same time as the hypoxia. The hypoxia may be 0.1%-10%, 0.1%-5%, 0.1%-2.5%, or 0.1%-1% oxygen, for example. In specific embodiments, the hypoxia occurs for a period of time that is at least or no more than between 30 minutes-3 days, although in some cases it may be less than 30 minutes or greater than 3 days. In specific embodiments, the hypoxia is sufficient to induce a 50% or greater induction of one or more of VEGF, HGF-1, BMP-2, LL-37, PD-L1, HLA-G, IL-10, or TGF-beta, for example. In some embodiments, the condition capable of enhancing regenerative activity is culture in the presence of an inflammatory stimuli, such as a Toll-like receptor agonist.

The Toll-like receptor agonist may comprise any composition capable of activating any Toll-like receptor. The Toll-like receptor may be TLR-1, TLR-2, TLR-3, TLR-4, TLR-5, TLR-6, TLR-7, TLR-8, and/or TLR-9. The Toll-like receptor agonist may comprise Pam3CSK4, HKLM, Poly:IC, LPS, Buprenorphine, Carbamazepine, Fentanyl, Levorphanol, Methadone, Cocaine, Morphine, Oxcarbazepine, Oxycodone, Pethidine, Glucuronoxylomannan from Cryptococcus, Morphine-3-glucuronide, lipoteichoic acid, β-defensin 2, a small molecular weight hyaluronic acid, fibronectin EDA, snapin, tenascin C, flagellin, FSL-1, imiquimod, ssRNA40/LyoVec, CpG oligonucleotide, ODN2006, agatolimod, or a combination thereof.

In some embodiments, the fibroblasts are associated with a device. In specific embodiments, the device comprises a scaffold including a flexible mesh impregnated with and carrying fibroblasts, regenerative stem cells, growth factors, and/or bone graft material. In some embodiments, the flexible mesh is comprised of one or more of natural hydrogel, synthetic hydrogel, and/or polymer. The fibroblast population can be in one of any positions relative to the device including the inside and/or outside and/or surface of the device. In some embodiments, the device is an implant. In some cases, the fibroblast population is separate from the device but is used in conjunction with the device, such as prior to, during, and/or subsequent to placement of the device in the oral cavity of the individual in need thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the disclosure, for example. Some embodiments of the disclosure may consist of or consist essentially of one or more elements, method steps, and/or methods of the disclosure. It is contemplated that any method or composition described herein can be implemented with respect to any other method or composition described herein. Embodiments discussed in the context of methods and/or compositions of the disclosure may be employed with respect to any other method or composition described herein. Thus, an embodiment pertaining to one method or composition may be applied to other methods and compositions of the disclosure as well.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

Embodiments of the disclosure include systems, methods, and compositions related to treatment or prevention of any kind of medical condition (disease, injury, combination thereof, and so forth) in the oral cavity of mammal, including a human, dog, cat, horse, and so forth. In particular embodiments, the medical condition concerns gum tissue, bone, or both in need of repair in the oral cavity of the individual.

In specific embodiments, the disclosure provides means of protecting against gingival loss, as well as generation or improvements of bone grafts with enhanced efficacy, through the utilization of fibroblasts and/or modified fibroblasts and/or derivatives thereof. In one embodiment, the ability of fibroblasts to produce regenerative, anti-inflammatory, and/or protease-inhibiting factors is utilized to reduce oral (including gingival) damage caused by any reason, including at least bacterial infection. In other embodiments, fibroblasts, modified fibroblasts, fibroblast-derived products, and/or conditioned media of fibroblasts are utilized to induce regeneration of gingival tissue. In other embodiments, fibroblasts are modified to possess enhanced expression of one or more molecules associated with regeneration of gingival tissue. It is an object of certain embodiments of the disclosure to utilize any fibroblasts or product thereof described herein to address the need for periodontal therapy, which is, in general, to preserve or maintain the dentition in a state of health and comfort throughout the life of an individual. In specific embodiments, the periodontal therapy utilizing the fibroblasts and/or derivatives thereof eliminates inflammation, arrests progression of periodontal disease, improves aesthetics, and generates an environment conducive to the maintenance of health of the individual. In the practice of certain methods of the disclosure, the individual may be enabled to play a more active role in self-treatment (e.g. brushing of teeth), rather than relying on a clinician.

In particular embodiments, the disclosure aims to treat gingival injury, which in addition to periodontal disease, refers to the loss or shrinkage of gingiva because of, for example, external trauma (e.g., a surgical procedure or wound, piercings, burns, radiation, blunt force, puncture, fracture, luxation, soft tissue injury, foreign body intrusion, gum trauma, dental and/or orthodontic malpractice, or an accident), an acute wound, a chronic wound, a scar, or a combination thereof. The individual may have a predisposition of spontaneous or induced fragile mucosa such as epidermolysis bullosa aquisita, autoimmune diseases with blisters, wounds or atrophy, such as pemphigus, a combination thereof, and others. The gingiva can age and/or be injured, leading to receding gums and potentially alveolar bone destruction because of abnormal tooth position, such as the crowding of teeth that gives rise to inadequate cover of one or more teeth by the jaw bone, inherited insufficient gingival tissues, acquired diseases with fragility, blistering, wounding or atrophy such as pemphigus and others, overaggressive brushing, periodontal diseases, improper flossing or brushing that allows bacteria to build up between the teeth and at plaques, self-induced vomiting or other eating disorders, shrinkage of gingival tissue because of ageing or by the use of chewing tobacco or the adverse effects of smoking, teeth grinding, scurvy and other nutritional deficiencies that affect proper growth or health of gums, and/or sensitivity to detergents. Gingival recessions may also be caused by gingivitis that then is associated with puffy red, swollen gums, bleeding, and bad breath. In addition, gingival injuries may incur by acute or chronic viral, mycotic or bacterial infections, chronic autoimmune inflammatory disease such as scleroderma and variants, Borelliosis infection, Lupus erythematosis and variants, Lichen ruber planus, and general aging (intrinsic aging). In one embodiment, the oral region in need of therapy or prevention is on part or all of the entire oral mucosa, including the soft and hard palate, or a selected portion of the jaws, such as individually affected teeth.

I. [0019] Fibroblasts or Production Thereof

In particular embodiments, fibroblasts are utilized to enhance a therapy for one or more oral medical conditions, including dental applications such as gum and/or bone therapy. The fibroblasts may or may not be obtained from a particular source and may or may not be manipulated or modified compared to fibroblasts in nature. Such modifications include exposure to one or more particular agents and/or exposure to one or more particular conditions, including prior to use and/or during use, and/or subsequent to medical intervention in the oral cavity of an individual in need thereof.

In some embodiments, fibroblasts are utilized in any methods of the disclosure, whereas in other cases derivatives of fibroblasts, such as exosomes from fibroblasts, conditioned media from culture of fibroblasts, or a combination thereof, are utilized. In some cases, fibroblasts are utilized with derivatives of fibroblasts. As used herein, “exosomes” encompass nanovesicles released from a variety of different cells, such as fibroblasts, including modified fibroblasts. These small vesicles may be derived from large multivesicular endosomes and secreted into the extracellular milieu. The precise mechanisms of exosome release/shedding remain unclear. They may form by invagination and budding from the limiting membrane of late endosomes, resulting in vesicles that contain cytosol and that expose the extracellular domain of membrane-bound cellular proteins on their surface. Using electron microscopy, studies have shown fusion profiles of multivesicular endosomes with the plasma membrane, leading to the secretion of the internal vesicles into the extracellular environment. In some cases, exosomes from fibroblasts are utilized following culture of the fibroblasts, including in some cases following exposure of the fibroblasts to one or more agents and/or conditions. Such agents and/or conditions may be any encompassed herein.

Disclosed are methods and compositions related to enhancement of therapeutic activity of fibroblasts in a regenerative capacity. In specific embodiments, fibroblasts are enhanced through stimulation of heme oxygenase (HO)-1 expression by one or more agents and/or conditions, and the stimulation may or may not be direct. Such enhancement may occur through (1) exposure of the fibroblasts to one or more agents and/or conditions that increase endogenous expression of HO-1; (2) exposure of the fibroblasts to exogenously provided HO-1; and/or (3) exposure of the fibroblasts to exogenously provided HO-1 upon expression of HO-1 from an exogenously provided vector in the fibroblasts that express HO-1, for example. In specific embodiments, the one or more agents includes carbon monoxide (CO), hypoxia, or a combination thereof. In certain embodiments when both CO and hypoxia are provided to the fibroblasts, the order of their exposure is particular. In some cases, they are provided to the fibroblasts at the same time, whereas in other cases they are provided to the fibroblasts at different times, and in some cases they are both provided to the cells at separate times and at the same time. In some cases, the CO is provided to the fibroblasts before the hypoxia, whereas in other cases the CO is provided to the fibroblasts after the hypoxia.

In some embodiments, there are methods of treating fibroblast cells to enhance their regenerative activity, comprising the steps of: a) optionally obtaining a fibroblast population (the fibroblasts may be obtained by another party or obtained from storage or commercially obtained, for example); b) priming the fibroblast population by exposure to hypoxia for a sufficient time period, for example to induce upregulation of hypoxia inducible factor (HIF)-1 alpha; and c) optionally subsequent to exposure to hypoxia, treating the fibroblasts with CO at a sufficient concentration and time period to induce upregulation of HO-1.

In particular embodiments, fibroblasts are enhanced by methods encompassed herein in their activity for cytokine secretion, upregulation of anti-apoptotic properties, and/or modulation of immunogenicity. In specific embodiments, the regenerative activity comprises enhanced production of one or more cytokines, such as cytokines selected from the group consisting of a) fibroblast growth factor (FGF)-1; b) FGF-2; c) FGF-5; d) FGF-12; e) IL-10; f) leukemia inhibitor factor; f) insulin growth factor; g) epidermal growth factor; h) vascular endothelial growth factor (VEGF); and i) a combination thereof.

Although the regenerative activity may be of any kind, in specific embodiments the regenerative activity comprises the ability for the fibroblasts to differentiate into any other type of cell including at least into chondrocytes; adipocytes; endothelial cells; neural cells; pancreatic islet cells, and/or osteocytes. In some embodiments, the regenerative activity comprises the ability for the fibroblasts to differentiate into any type of cell found in any oral tissue.

In some embodiments, a regenerative activity comprises an ability of the fibroblasts to produce a conditioned media that is mitogenic for any type of cell, including at least fibroblasts; mesenchymal stem cells; notochord cells; and/or chondrocytic progenitors cells, for example.

In particular embodiments, fibroblasts have an enhanced regenerative activity following exposure to one or more certain conditions, including exposure of a condition for a certain period of time or other form of parameter for the condition. In specific embodiments, fibroblasts are exposed to a hypoxic environment. As an example, incubation of fibroblasts in a hypoxic environment may be performed under conditions of at least or no more than 0.1%-10% oxygen; 0.1%-9%; 0.1%-8%; 0.1%-7%; 0.1%-6%; 0.1%-5%; 0.1%-4%; 0.1%-3%; 0.1%-2%; 0.1%-1%; 0.1%-0.75%; 0.1%-0.5%; 0.25%-10%; 0.25%-9%; 0.25%-8%; 0.25%-7%; 0.25%-6%; 0.25%-5%; 0.25%-4%; 0.25%-3%; 0.25%-2%; 0.25%-1%; 0.25%-0.75%; 0.25%-0.5%; 0.5%-10%; 0.5%-9%; 0.5%-8%; 0.5%-7%; 0.5%-6%; 0.5%-5%; 0.5%-4%; 0.5%-3%; 0.5%-2%; 0.5%-1%; 0.5%-0.75%; 0.75%-10%; 0.75%-9%; 0.75%-8%; 0.75%-7%; 0.75%-6%; 0.75%-5%; 0.75%-4%; 0.75%-3%; 0.75%-2%; 0.75%-1%; 1%-9%; 1%-8%; 1%-7%; 1%-6%; 1%-5%; 1%-4%; 1%-3%; 1%-2%; 2%-9%; 2%-8%; 2%-7%; 2%-6%; 2%-5%; 2%-4%; 2%-3%; 3%-9%; 3%-8%; 3%-7%; 3%-6%; 3%-5%; 3%-4%; 4%-9%; 4%-8%; 4%-7%; 4%-6%; 4%-5%; 5%-9%; 5%-8%; 5%-7%; 5%-6%; 6%-9%; 6%-8%; 6%-7%; 7%-9%; 7%-8%; or 8%-9%, 0.1%, 0.2%, 0.3%, 0.4%, 0.5%, 0.6%, 0.7%, 0.8%, 0.9%, 1.0%, 1.25%, 1.5%, 1.75%, 2.0%, 2.25%, 2.5%, 2.75%, 3%, 3.25%, 3.5%, 3.75%, 4.0%, 4.25%, 4.5%, 4.75%, 5%, 5.25%, 5.5%, 5.75%, 6%, 6.25%, 6.5%, 6.75%, 7.25%, 7.5%, 7.75%, 8%, 8.25%, 8.5%, 8.75%, 9%, or 10%, for example.

In some cases exposure of the fibroblasts to the hypoxic environment occurs for a certain period of time, and this time may or may not be continual. In specific embodiments, incubation of fibroblasts in a hypoxic environment occurs for a period of time, such as at least or no more than between 30 minutes (min)-3 days; 30 min-2 days; 30 min-1 days; 30 min-18 hours (hrs); 30 min-12 hrs; 30 min-8 hrs; 30 min-6 hrs; 30 min-4 hrs; 30 min-2 hrs; 30 min-1 hr; 45 min-3 days; 45 min-2 days; 45 min-1 days; 45 min-18 hrs; 45 min-12 hrs; 45 min-8 hrs; 45 min-6 hrs; 45 min-4 hrs; 45 min-2 hrs; 45 min-1 hr; 60 min-3 days; 60 min-2 days; 60 min-1 days; 60 min-18 hrs; 60 min-12 hrs; 60 min-8 hrs; 60 min-6 hrs; 60 min-4 hrs; 60 min-2 hrs; 60 min-1 hr; 2 hrs-3 days; 2 hrs-2 days; 2 hrs-1 days; 2 hrs-18 hrs; 2 hrs-12 hrs; 2 hrs-8 hrs; 2 hrs-6 hrs; 2 hrs-4 hrs; 2 hrs-3 hrs; 6 hrs-3 days; 6 hrs-2 days; 6 hrs-1 day; 6 hrs-18 hrs; 6 hrs-12 hrs; 6 hrs-8 hrs; 8 hrs-3 days; 8 hrs-2 days; 8 hrs-1 day; 8 hrs-18 hrs; 8 hrs-12 hrs; 8 hrs-10 hrs; 12 hrs-3 days; 12 hrs-2 days; 12 hrs-1 day; 12 hrs-18 hrs; 18 hrs-3 days; 18 hrs-2 days; 18 hrs-1 day; 1 day-3 days; or 2 days-3 days, for example. In specific embodiments, such incubation occurs for 1-8; 1-7; 1-6; 1-5; 1-4; 1-3; 1-2; 2-8; 2-7; 2-6; 2-5; 2-4; 2-3; 3-8; 3-7; 3-6; 3-5; 3-4; 4-8; 4-7; 4-6; 4-5; 5-8; 5-7; 5-6; 6-8; 6-7; or 7-8 hours, for example.

In certain embodiments, the fibroblasts are exposed to certain levels of CO. In particular embodiments, incubation of the fibroblasts with CO is performed at a concentration of at least or no more than 1-500 parts per million (ppm); 1-400 ppm; 1-300 ppm; 1-250 ppm; 1-200 ppm; 1-175 ppm; 1-150 ppm; 1-125 ppm; 1-100 ppm; 1-75 ppm; 1-50 ppm; 1-25 ppm; 1-10 ppm; 10-500 ppm; 10-400 ppm; 10-300 ppm; 10-250 ppm; 10-200 ppm; 10-150 ppm; 10-125 ppm; 10-100 ppm; 10-75 ppm; 10-50 ppm; 10-25 ppm; 25-500 ppm; 25-400 ppm; 25-300 ppm; 25-250 ppm; 25-200 ppm; 25-175 ppm; 25-150 ppm; 25-125 ppm; 25-100 ppm; 25-75 ppm; 25-50 ppm; 50-500 ppm; 50-400 ppm; 50-300 ppm; 50-200 ppm; 50-175 ppm; 50-150 ppm; 50-125 ppm; 50-100 ppm; 50-75 ppm; 75-500 ppm; 75-400 ppm; 75-300 ppm; 75-250 ppm; 75-225 ppm; 75-200 ppm; 75-175 ppm; 75-150 ppm; 75-125 ppm; 75-100 ppm; 100-500 ppm; 100-400 ppm; 100-300 ppm; 100-200 ppm; 100-150 ppm; 100-125 pm; 125-500 ppm; 125-400 ppm; 125-300 ppm; 125-275 ppm; 125-200 ppm; 125-175 ppm; 125-150 ppm; 150-500 ppm; 150-400 ppm; 150-300 ppm; 150-200 ppm; 150-175 ppm; 175-500 ppm; 175-400 ppm; 175-300 ppm; 175-275 ppm; 175-250 ppm; 175-225 ppm; 175-200 ppm; 200-500 ppm; 200-400 ppm; 200-300 ppm; 200-250 ppm; 250-500 ppm; 250-400 ppm; 250-300 ppm; 250-275 ppm; 300-500 ppm; 300-400 ppm; or 400-500 ppm, for example. In specific cases, incubation of fibroblasts with CO is performed at a concentration of 75, 100, 125, 150, 175, 200, 225, 250, 275, 300, or 325 or more parts per million (ppm) for 1, 2, 3, 4, 5, 6, 7, 8, 9, or 10 or more hours.

In particular embodiments, the fibroblasts are exposed to a particular level of CO as described above for a particular period of time, such as at least or no more than between 30 minutes (min)-3 days; 30 min-2 days; 30 min-1 days; 30 min-18 hours (hrs); 30 min-12 hrs; 30 min-8 hrs; 30 min-6 hrs; 30 min-4 hrs; 30 min-2 hrs; 30 min-1 hr; 45 min-3 days; 45 min-2 days; 45 min-1 days; 45 min-18 hrs; 45 min-12 hrs; 45 min-8 hrs; 45 min-6 hrs; 45 min-4 hrs; 45 min-2 hrs; 45 min-1 hr; 60 min-3 days; 60 min-2 days; 60 min-1 days; 60 min-18 hrs; 60 min-12 hrs; 60 min-8 hrs; 60 min-6 hrs; 60 min-4 hrs; 60 min-2 hrs; 60 min-1 hr; 2 hrs-3 days; 2 hrs-2 days; 2 hrs-1 days; 2 hrs-18 hrs; 2 hrs-12 hrs; 2 hrs-8 hrs; 2 hrs-6 hrs; 2 hrs-4 hrs; 2 hrs-3 hrs; 6 hrs-3 days; 6 hrs-2 days; 6 hrs-1 day; 6 hrs-18 hrs; 6 hrs-12 hrs; 6 hrs-8 hrs; 8 hrs-3 days; 8 hrs-2 days; 8 hrs-1 day; 8 hrs-18 hrs; 8 hrs-12 hrs; 8 hrs-10 hrs; 12 hrs-3 days; 12 hrs-2 days; 12 hrs-1 day; 12 hrs-18 hrs; 18 hrs-3 days; 18 hrs-2 days; 18 hrs-1 day; 1 day-3 days; or 2 days-3 days, for example. In specific embodiments, such incubation occurs for 1-8; 1-7; 1-6; 1-5; 1-4; 1-3; 1-2; 2-8; 2-7; 2-6; 2-5; 2-4; 2-3; 3-8; 3-7; 3-6; 3-5; 3-4; 4-8; 4-7; 4-6; 4-5; 5-8; 5-7; 5-6; 6-8; 6-7; or 7-8 hours, for example.

In particular embodiments, the cells are treated with hypoxia prior to treatment with CO, although in other embodiments the cells are treated with hypoxia during and/or after treatment with CO. In certain embodiments, there is a synergistic effect on the fibroblasts (for example, synergistic growth factor production by the fibroblasts) upon sequential exposure of the cells to hypoxia followed by CO treatment.

In particular embodiments, the disclosure provides methods of augmenting therapeutic activity of fibroblasts through modification of culture conditions for the fibroblasts. In specific embodiments, there are previously undisclosed synergies in terms of stimulation of regenerative activity by culture of fibroblasts in hypoxia, together with low concentrations of carbon monoxide. In one embodiment, fibroblasts are exposed to one or more agents and/or one or more culture conditions for the purpose of the fibroblasts producing a conditioned media comprising one or more factors and/or other agent(s) for a therapeutic application.

In specific cases, the fibroblasts (following exposure to one or more particular agents and/or culture conditions) are utilized as a source of conditioned media; in certain cases the conditioned media comprises one or more particular growth factors or other agent(s). In specific embodiments, conditioned media produced by the fibroblasts is stored for future use or the media may be used without first being stored. In some cases, the fibroblasts produce a conditioned media and the media is provided to the individual from which the fibroblasts were obtained. In other cases the fibroblasts produce a conditioned media and the media is provided to the individual other than the one from which the fibroblasts were obtained. The conditioned media may be generated in a patient-specific context (using autologous fibroblasts) or universal donor context (using allogenic fibroblasts).

In specific embodiments, the fibroblast-conditioned media may be utilized for any application, including acceleration of wound healing, induction of angiogenesis, and/or for cosmetic means, as examples. In specific embodiments, fibroblasts are exposed to conditions augmenting regenerative properties and administered to an individual as a supplement or substitute to stem cells, including mesenchymal stem cells. In one embodiment, fibroblasts are injected at a particular site in an individual, such as at an oral location in need of regeneration, for stimulation of regeneration. Augmentation of fibroblast regenerative activity as disclosed herein may be achieved by a two-step process involving an initial exposure to hypoxia, followed by exposure to conditions stimulating HO-1, such as CO, in certain cases.

In one aspect of the disclosure, potency of the conditioned media product from the fibroblasts may be analyzed. In specific embodiments, the conditioned media may be analyzed for assessing protein production, for example. Such assays are well-known to one of skill in the art.

The conditioned media may be analyzed for anti-inflammatory activity, for example the conditioned media may have anti-inflammatory activity so that it can be used as an anti-inflammatory agent or with one or more other anti-inflammatory agents. For quantification of anti-inflammatory activity, the term “inflammation” will be understood by those skilled in the art to include any condition characterized by a localized or a systemic protective response, which may be elicited by, for example, tooth loss, tooth decay, tooth impaction, gum disease, periodontitis, abscess, mouth ulcer, inflammation, leukoplakia, halitosis, infection, microbiome dysbiosis, bacterial microfilm, or a combination thereof. Any such response, which may serve to destroy, dilute or sequester both the injurious agent and the injured tissue, may be manifested by, for example, heat, swelling, pain, redness, dilation of blood vessels and/or increased blood flow, invasion of the affected area by white blood cells, loss of function and/or any other symptoms known to be associated with inflammatory conditions. The term “inflammation” will thus also be understood to include any inflammatory disease, disorder or condition per se, any condition that has an inflammatory component associated with it, and/or any condition characterized by inflammation as a symptom, including, inter alia, acute, chronic, ulcerative, specific, allergic and necrotic inflammation, and other forms of inflammation known to those skilled in the art. The term thus also includes, for the purposes of this disclosure, inflammatory pain and/or fever caused by inflammation.

In one embodiment, conditioned media is generated in an ex-vivo extracorporeal setting. Specifically, fibroblast cells of interest are grown extracorporeally, such as on and/or in one or more substrates. In a specific embodiment, the cells are grown on the outside of a hollow-fiber filter, for example that is connected to a continuous extracorporeal system. The hollow-fiber system contains pores in the hollow fiber of sufficient size so as to allow exchange of proteins between circulating blood cells and cultured cells on the outside of the hollow fibers, without interchange of host cells with the fibroblast cells. In one embodiment, fibroblast-conditioned media is used in combination with one or more immune suppressive agents to augment its activity.

While fibroblast-conditioned media may or may not be used alone for treatment and/or maintenance of disease remission, in some embodiments, administration of one or more immune suppressive agents may be utilized in certain methods. Additionally, an immune suppressive agent may be useful for induction therapy. Depending on disease and response desired, it will be known to one of skill in the art to choose from various immune suppressive agents. For example, some immune suppressive agents, such as anti-CD52 antibodies, may be used when a systemic depletion of T and B cells is desired, whereas agents that concurrently stimulate T regulatory cell activity, such as Rapamycin, may be desired in other cases. The skilled practitioner is guided to examples of several agents that are known in the art for causing immune suppression, which include cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-alpha inhibitors, TNF-alpha sequestration agents, and methotrexate. More specifically, anti-inflammatory agents may comprise one or more of, e.g., anti-TNF-alpha, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, or a combination thereof.

The fibroblasts utilized in the disclosure may be generated, in one embodiment, by outgrowth from a biopsy of the recipient's own skin (in the case of autologous preparations), or skin of one or more healthy donors (for allogeneic preparations). In some cases, a combination of autologous and allogeneic cells are utilized. In some embodiments fibroblasts are used from young donors, although the donor may be of any age, including newborn, infant, child, adolescent, adult, or elderly. In another embodiment, fibroblasts are transfected with one or more genes. In specific examples, the fibroblasts are transfected with one or more genes to allow for enhanced growth and overcoming of the Hayflick limit. Examples of such genes includes telomerase, hTERT, and/or one or more oncogenes, such as RAS, c-myc, RAF, or bcr-able. Subsequent to derivation of cells, the fibroblasts may be expanded in culture using standard cell culture techniques. Skin tissue (dermis and epidermis layers) may be biopsied from a subject's post-auricular area, in some cases. In one embodiment, the starting material is comprised of multiple 3-mm punch skin biopsies collected using standard aseptic practices. The biopsies may be collected by a medical practitioner and placed into a vessel comprising a suitable buffer, such as a vial comprising sterile phosphate buffered saline (PBS). The biopsies may be transported prior to use, such as transported at a suitable temperature (for example, 2-8° C.). The biopsies may be transported suitably to a manufacturing facility. In one embodiment, after arrival at the manufacturing facility, a biopsy may be inspected and, upon acceptance, transferred directly to a manufacturing area.

Upon initiation of the process of preparing the cells, the biopsy tissue may be washed, such as washed prior to enzymatic digestion. After washing, enzymes may be exposed to the tissue, such as mixtures of collagenase and/or neutral protease enzymes (for example, Liberase™ Digestive Enzyme Solution (Lonza Walkersville, Inc. (Walkersville, Md.) or Roche Diagnostics Corp. (Indianapolis, Ind.)) being added to the tissue with or without mincing, and the biopsy tissue is then incubated at suitable temperature and duration, such as 37.0±2° C. for one hour. Time of biopsy tissue digestion is a process parameter that can affect the viability and growth rate of cells in culture. Alternatively to Liberase™, other commercially available collagenases may be used, such as Serva Collagenase NB6 (Helidelburg, Germany). After digestion, suitable media may be provided to the digested material, such as Initiation Growth Media (IMDM, GA, 10% Fetal Bovine Serum (FBS)) that may be added to neutralize the enzyme. The cells may be pelleted, such as by centrifugation, and re-suspended in 5.0 mL Initiation Growth Media. Alternatively, centrifugation is not performed, with full inactivation of the enzyme occurring by the addition of media (such as Initiation Growth Media) only. Initiation Growth Media may be added prior to seeding of the cell suspension into suitable vessel, such as a T-175 cell culture flask for initiation of cell growth and expansion. In some cases, T-75, T-150, T-185 or T-225 flask can be used in place of the T-75 flask. Cells are incubated at 37±2.0° C. with 5.0±1.0% CO₂ and fed with fresh media, such as Complete Growth Media, at a suitable interval, such as every three to five days. Feeds in the process may be performed by removing a fraction (such as about half) of the media (such as Complete Growth Media) and replacing the same volume with fresh media. Alternatively, full feeds can be performed. In one embodiment, cells are not left in the flask greater than a certain number of days prior to passaging, such as about 30 days and may be left at least 24 hours prior to passaging. Confluence is monitored throughout the process to ensure adequate seeding densities during culture splitting. When cell confluence is greater than or equal to a certain percentage (such as about 40%-about 70%) in the vessel, the cells may be passaged by removing the spent media, washing the cells, and treating with Trypsin-EDTA to release adherent cells in the flask into the solution. Cells may then be trypsinized and seeded into a suitable vessel (T-500 flask, for example) for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology may be evaluated at each passage and prior to harvest to monitor the culture purity throughout the process. Morphology may be evaluated by comparing the observed sample with visual standards for morphology examination of cell cultures, for example. The cells display typical fibroblast morphologies when growing in cultured monolayers. Cells may display either an elongated, fusiform or spindle appearance with slender extensions, or appear as larger, flattened stellate cells that may have cytoplasmic leading edges. A mixture of these morphologies may also be observed. Fibroblasts in less confluent areas can be similarly shaped, but randomly oriented. The presence of keratinocytes in cell cultures may also be evaluated. Keratinocytes appear round and irregularly shaped and, at higher confluence, they appear organized in a cobblestone formation. At lower confluence, keratinocytes are observable in small colonies. Cells are incubated at 37±2.0° C. with 5.0±1.0% CO₂ and passaged every three to five days in the T-500 flask and every five to seven days in the ten layer cell stack (10CS). Cells should not remain in the T-500 flask for more than 10 days prior to passaging, in some embodiments. Quality Control (QC) release testing for safety of the Bulk Drug Substance includes sterility and endotoxin testing. When cell confluence in the T-500 flask is approximately 95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% CO₂ and fed with fresh Complete Growth Media every five to seven days. Cells should not remain in the 10CS for more than 20 days prior to passaging, in particular embodiments. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting may be performed by removing the spent media, washing the cells, treating with Trypsin-EDTA to release adherent cells into the solution, and adding additional Complete Growth Media to neutralize the trypsin. Cells may be collected by centrifugation, re-suspended, and in-process QC testing performed to determine total viable cell count and cell viability.

In some embodiments, when large numbers of cells are required after receiving cell count results from the primary 10 CS harvest, an additional passage into multiple cell stacks (up to four 10 CS) may be performed. For additional passaging, cells from the primary harvest are added to a 2 L media bottle containing fresh Complete Growth Media. Resuspended cells are added to multiple cell stacks and incubated at 37±2.0° C. with 5.0±1.0% CO₂. The cell stacks are fed and harvested as described above, except cell confluence is 80% or higher prior to cell harvest, in certain embodiments. The harvest procedure may be the same as described for the primary harvest above. A mycoplasma sample from cells and spent media is collected, and cell count and viability performed as described for the primary harvest above. The method decreases or eliminates immunogenic proteins by avoiding their introduction from animal-sourced reagents. To reduce process residuals, cells are cryopreserved in protein-free freeze media, then thawed and washed prior to prepping the final injection to further reduce remaining residuals. If additional Drug Substance is needed after the harvest and cryopreservation of cells from additional passaging is complete, aliquots of frozen Drug Substance—Cryovial are thawed and used to seed 5 CS or 10 CS culture vessels. Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest.

At the completion of culture expansion, the cells are harvested and washed, then formulated to contain 1.0-2.7×10⁷ cells/mL, with a target of 2.2×10⁷ cells/mL. Alternatively, the target can be adjusted within the formulation range to accommodate different indication doses. The drug substance comprises a population of viable, autologous human fibroblast cells suspended in a suitable medium, such as a cryopreservation medium comprised of Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. In addition to cell count and viability, purity/identity of the Drug Substance is performed and must confirm the suspension contains 98% or more fibroblasts. The usual cell contaminants include keratinocytes. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin .beta.4 chain, is a 205 kDa transmembrane glycoprotein which associates with integrin.alpha.6 chain (CD49f) to form the alpha6/beta4 complex. This complex has been shown to act as a molecular marker for keratinocyte cells (Adams and Watt 1991).

Antibodies to CD 104 protein bind to 100% of human fibroblast cells. Cell count and viability is determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead. The method was custom developed specifically for use in determining purity/identity of autologous cultured fibroblasts. Alternatively, cells can be passaged from either the T-175 flask (or alternatives) or the T-500 flask (or alternatives) into a spinner flask containing microcarriers as the cell growth surface. Microcarriers are small bead-like structures that are used as a growth surface for anchorage dependent cells in suspension culture. They are designed to produce large cell yields in small volumes. In this apparatus, a volume of Complete Growth Media ranging from 50 mL-300 mL is added to a 500 mL, IL or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37±2.0° C. with 5.0±1.0% CO₂ incubator to allow for adherence of cells to the carriers. After the attachment period, the speed of the spin plate is increased (30-120 RPM). Cells are fed with fresh Complete Growth Media every one to five days, or when media appears spent by color change. Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsinizationtrypsiziation and reseeding. Alternatively, if enough cells are produced from the initial T-175 or T-500 flask, the cells can be directly seeded into the scale-up amount of microcarriers. After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA. Microcarriers used within the disposable spinner flask may be made from poly blend such as BioNOC II® (Cesco Bioengineering, distributed by Bellco Biotechnology, Vineland, N.J.) and FibraCel® (New Brunswick Scientific, Edison, N.J.), gelatin, such as Cultispher-G (Percell Biolytica, Astrop, Sweden), cellulose, such as Cytopore™. (GE Healthcare, Piscataway, N.J.) or coated/uncoated polystyrene, such as 2D MicroHex™. (Nunc, Weisbaden, Germany), Cytodex® (GE Healthcare, Piscataway, N.J.) or Hy-Q Sphere™ (Thermo Scientific Hyclone, Logan, Utah).

In another embodiment, cells can be processed on poly blend 2D microcarriers such as BioNOC II® and FibraCel® using an automatic bellow system, such as FibraStage™ (New Brunswick Scientific, Edison, N.J.) or BelloCell® (Cesco Bioengineering, distributed by Bellco Biotechnology, Vineland, N.J.) in place of the spinner flask apparatus. Cells from the T-175 (or alternatives) or T-500 flask (or alternatives) may be passaged into a bellow bottle containing microcarriers with the appropriate amount of Complete Growth Media, and placed into the system. The system pumps media over the microcarriers to feed cells, and draws away media to allow for oxygenation in a repeating fixed cycle. Cells are monitored, fed, washed and harvested in the same sequence as described above. Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells are expanded in a cell tower, consisting of a stack of disks capable of supporting anchorage-dependent cell attachment. The system automatically circulates media and performs trypsinization for harvest upon completion of the cell expansion stage.

Alternatively, the ACE system can be a scaled down, single lot unit version comprised of a disposable component that consists of cell growth surface, delivery tubing, media and reagents, and a permanent base that houses mechanics and computer processing capabilities for heating/cooling, media transfer and execution of the automated programming cycle. Upon receipt, each sterile irradiated ACE disposable unit will be unwrapped from its packaging and loaded with media and reagents by hanging pre-filled bags and connecting the bags to the existing tubing via aseptic connectors. The process continues as follows: a) Inside a biological safety cabinet (BSC), a suspension of cells from a biopsy that has been enzymatically digested is introduced into the “pre-growth chamber” (small unit on top of the cell tower), which is already filled with Initiation Growth Media containing antibiotics. From the BSC, the disposable would be transferred to the permanent ACE unit already in place; b) After approximately three days, the cells within the pre-growth chamber are trypsinized and introduced into the cell tower itself, which is pre-filled with Complete Growth Media. Here, the “bubbling action” caused by CO₂ injection force the media to circulate at such a rate that the cells spiral downward and settle on the surface of the discs in an evenly distributed manner; c) For approximately seven days, the cells are allowed to multiply. At this time, confluence will be checked (method unknown at time of writing) to verify that culture is growing. Also at this time, the Complete Growth Media will be replaced with fresh Complete Growth Media. CGM may be replaced at suitable intervals, such as every seven days for three to four weeks. At the end of the culture period, the confluence is checked once more to verify that there is sufficient growth to possibly yield the desired quantity of cells for the intended treatment; d) If the culture is sufficiently confluent, it is harvested. The spent media (supernatant) is drained from the vessel. PBS will then is pumped into the vessel (to wash the media, FBS from the cells) and drained almost immediately. Trypsin-EDTA is pumped into the vessel to detach the cells from the growth surface. The trypsin/cell mixture is drained from the vessel and enter the spin separator. Cryopreservative is pumped into the vessel to rinse any residual cells from the surface of the discs, and be sent to the spin separator as well. The spin separator collects the cells and then evenly resuspend the cells in the shipping/injection medium. From the spin separator, the cells will be sent through an inline automated cell counting device or a sample collected for cell count and viability testing via laboratory analyses. Once a specific number of cells has been counted and the proper cell concentration has been reached, the harvested cells are delivered to a collection vial that can be removed to aliquot the samples for cryogenic freezing.

In another embodiment, automated robotic systems may be used to perform cell feeding, passaging, and harvesting for the entire length or a portion of the process. Cells can be introduced into the robotic device directly after digest and seed into the T-175 flask (or alternative). The device may have the capacity to incubate cells, perform cell count and viability analysis and perform feeds and transfers to larger culture vessels. The system may also have a computerized cataloging function to track individual lots. Existing technologies or customized systems may be used for the robotic option.

In one embodiment, fibroblasts are pre-activated before exposure to hypoxia/carbon monoxide by contact with one or more growth factors, such as in a mixture, including a growth factor containing mixture. The mixture may comprise growth factor(s) selected from the group consisting of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF), and mixtures thereof. In specific embodiments, the growth factors are transforming growth factors (TGF), platelet-derived growth factors (PDGF), fibroblast growth factors (FGF), or mixtures thereof. In particular embodiments, the growth factor(s) are selected from the group consisting of transforming growth factors beta (TGF-beta), platelet-derived growth factors BB (PDGF-BB), basic fibroblast growth factors (bFGF), and mixtures thereof. In another embodiment of the disclosure, growth factor—comprising compositions are provided simultaneously with, or subsequent to, delivery of fibroblasts, either or both of which may be injected. The fibroblasts may be autologous, allogeneic, or xenogeneic. In some embodiments a platelet plasma composition is administered together with the fibroblasts or separately from the fibroblasts, for example subsequent to administration of the fibroblasts. A platelet plasma composition may comprise, consist essentially of, or consist of platelets and plasma and may be derived from bone marrow or peripheral blood, for example. The present disclosure may use platelet plasma compositions from either or both of these sources, and either platelet plasma composition may be used to regenerate any oral tissue in need thereof. Further, a platelet plasma composition may be used with or without concentrated bone marrow (BMAC). By way of example, when inserted into the annulus, 0.05-2.0 cc of platelet plasma composition may be used, and when inserted into the nucleus, 0.05-3.0 cc of the platelet plasma composition may be used[NRF1]. Platelets are non-nucleated blood cells that as noted above are found in bone marrow and peripheral blood. They have several important functions such as controlling bleeding and tissue healing. As persons of ordinary skill in the art are aware, the ability to promote tissue healing is due to the many growth factors that they produce, including platelet-derived growth factor (PDGF), transforming growth factor beta (TGF-beta), fibroblast growth factor (FGF), insulin-like growth factor-1 (IGF-1), connective tissue growth factor (CTGF) and/or vascular endothelial growth factor (VEGF).

In particular embodiments, the fibroblasts are cultured prior to use. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time. Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm2 in culture. Preferably these cell numbers are produced within 80, 70, or 60 days or less. A cell line is a population of cells formed by one or more subcultivations of a primary cell culture. In some embodiments of the disclosure, fibroblasts are utilized as cell lines. In other embodiments, fibroblasts are utilized as primary cells. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

When referring to cultured cells, including fibroblast cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

As used herein, the term “growth medium” generally refers to a medium sufficient for the culturing of umbilicus-derived cells. In particular, a growth medium for the culturing of the cells herein may comprise Dulbecco's Modified Essential Media (also abbreviated DMEM herein). In some embodiments, the growth medium comprises DMEM-low glucose (also DMEM-LG herein) (Invitrogen, Carlsbad, Calif.). The DMEM-low glucose may be supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to growth medium.

Also relating to the present disclosure, the term standard growth conditions, as used herein refers to culturing of cells at 37° C., in a standard atmosphere comprising 5% CO₂. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

II. Methods of Use of the Fibroblasts

Embodiments of the disclosure include fibroblasts (including at least prepared or modified), fibroblast-derived products, and/or fibroblast-conditioned media for an oral (including dental) therapeutic or preventative purpose for an individual. In some cases, conditioned media and/or exosomes from the fibroblasts are provided to an individual in addition to or alternative to the prepared fibroblasts. In some embodiments, the methods include recognition of the need for use of the fibroblasts for the oral therapeutic or preventative purpose.

Certain embodiments relate to methods and devices for preventing or treating gum disease. In one embodiment of the disclosure, the gum disease is caused by a bacterial source, or associated with a bacterial microfilm or microbiome dysbiosis in the oral cavity. In another embodiment, the disclosure encompasses methods and devices for preventing or treating gum disease associated with enhanced production of proteases or reduced production of protease inhibitors in the gingival tissue. In particular cases, the protease inhibitors are tissue inhibitors of metalloproteases.

In some embodiments, following preparation of the fibroblasts, including as encompassed herein such that their regenerative activity is enhanced, a suitable number of fibroblasts are provided to one or more individuals in need thereof, including multiple oral deliveries where necessary. In some embodiments, the disclosure includes methods of generating a therapeutic product through growth of fibroblast populations in a liquid media and providing such to one or more individuals in need thereof. In one embodiment, there are methods of generating a medicament comprising prepared fibroblasts and/or conditioned media and/or fibroblast-derived products (such as exosomes) useful for the treatment of at least one medical condition, including at least tooth loss, tooth decay, tooth impaction, gum disease, periodontitis, abscess, mouth ulcer, inflammation, leukoplakia, halitosis, infection, microbiome dysbiosis, bacterial microfilm, cancer, or a combination thereof. In specific embodiments, the fibroblasts are prepared through culturing of the fibroblasts such that they are exposed to hypoxia and/or CO at suitable levels and suitable exposures. In some cases, the hypoxia condition may first be provided to the fibroblasts followed by carbon monoxide, both at a concentration and time of exposure sufficient in specific cases to stimulate HIF-1 alpha and heme-oxygenase, for example respectively.

Many types of media may be used by one of skill in the art to prepare the fibroblasts, conditioned media, and/or exosomes. In one embodiment, a media is selected from a group comprising of alpha MEM, DMEM, RPMI, Opti-MEM, IMEM, and AIM-V. Cells may be cultured in a variety of media for expansion that may or may not contain fetal calf serum or other growth factors; however, for collection of therapeutic supernatant, in a particular embodiment, the cells are transferred to a media substantially lacking serum. In some embodiments, the conditioned medium may be provided directly to an individual in need of treatment. It is well known in the art that preparation of the conditioned medium before administration may be performed by various means. For example, the conditioned medium may be filter sterilized, or in some conditions concentrated. In a particular embodiment, the conditioned medium is administrated for induction of regenerative activities, alone, or in combination with cells possessing regenerative properties.

In some embodiments, the prepared cells and/or conditioned media and/or exosomes are utilized for methods of promoting healing of oral wounds by administering cultured fibroblasts and/or conditioned media and/or exosomes therefrom. Examples of wounds include blunt force, puncture, fracture, luxation, soft tissue injury, burn, foreign body intrusion, gum trauma, dental and/or orthodontic malpractice or a combination thereof.

The prepared fibroblasts, which in specific embodiments are HO-1 augmented, are useful for a variety of therapeutic indications, in many situations analogous for which stem cells, including mesenchymal stem cells, would be useful. Examples of the amount of cells that may be provided to an individual include a range of 10,000/kg to 300 million/kg.

In some embodiments, between about 10⁵ and about 10¹³ cells per 100 kg are administered to an individual per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

In some embodiments, conditioned media is used as an active ingredient for a pharmaceutical formulation, either alone or with fibroblasts. This may comprise administration of the hypoxic/carbon monoxide fibroblast cell-conditioned media therapeutic agent alone, but in some cases comprises administration by way of known pharmaceutical formulations, including tablets, capsules or elixirs for oral administration, suppositories for rectal administration, sterile solutions or suspensions for parenteral or intramuscular administration, liposomal or encapsulated formulations, formulations wherein a therapeutic agent is alone or conjugated to a delivery agent or vehicle, and the like. It will be appreciated that therapeutic entities of the disclosure (including prepared fibroblasts) may be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Powell et al. “Compendium of excipients for parenteral formulations” PDA J Pharm Sci Technol 52:238-311 (1998) and the citations therein for additional information related to excipients and carriers well known to pharmaceutical chemists. In one embodiment of the disclosure, one or more agents of the disclosure are nanoencapsulated into nanoparticles for delivery. The nanoencapsulation material may be biodegradable or nondegradable. The nanoencapsulation materials may be made of synthetic polymers, natural polymers, oligomers, or monomers. Synthetic polymers, oligomers, and monomers include those derived from polyalkyleneoxide precursor molecules, such as poly(ethylene oxide) (PEO), poly(ethylene glycol) (PEG) and copolymers with poly(propylene oxide) (PEG-co-PPO), poly (vinyl alcohol) (PVA), poly(vinylpyrrolidone) (PVP), poly(ethyloxazoline) (PEOX), polyaminoacids, and pseudopolyamino acids, and copolymers of these polymers. Sawhney et al., Macromolecules 26:581-587 (1993). Copolymers may also be formed with other water-soluble polymers or water insoluble polymers, provided that the conjugate is water soluble. An example of a water-soluble conjugate is a block copolymer of polyethylene glycol and polypropylene oxide, commercially available as a Pluronic™ surfactant (BASF). Natural polymers, oligomers and monomers include proteins, such as fibrinogen, fibrin, gelatin, collagen, elastin, zein, and albumin, whether produced from natural or recombinant sources, and polysaccharides, such as agarose, alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageen. These polymers are merely exemplary of the types of nanoencapsulation materials that can be utilized and are not intended to represent all the nanoencapsulation materials within which entrapment is possible. In one embodiment, the therapeutic agent is administered in a topical formulation. Topical formulations are useful in the treatment of conditions associated with dermal diseases. For example, topical administration of fibroblast cell-conditioned media and/or the prepared fibroblasts may be performed for the treatment of psoriasis, scleroderma, or acne. Topical forms of administration may consist of, for example, aqueous and nonaqueous gels, creams, multiple emulsions, microemulsions, liposomes, ointments, aqueous and nonaqueous solutions, lotions, aerosols, skin patches, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Topical formulations of the disclosure may include a substance that is capable of delivering the other components of the formulation to any oral tissue with acceptable application or absorption of those components by the oral tissue. The carrier will typically include a solvent to dissolve or disperse the therapeutic agent, and, optionally one or more excipients or other vehicle ingredients. Carriers useful in accordance with the topical formulations of the present disclosure may include, by way of non-limiting example, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). Topical formulations of the disclosure are prepared by mixing a compound of the disclosure with a topical carrier according to well-known methods in the art, for example, methods provided by standard reference texts e.g., Remington: The Science and Practice of Pharmacy, 1577-1591, 1672-1673, 866-885 (Alfonso R. Gennaro ed. 19th ed. 1995); and Ghosh et al., Transdermal and Topical Drug Delivery Systems (1997). In other embodiments, moisturizers or humectants, sunscreens, fragrances, dyes, and/or thickening agents such as paraffin, jojoba, PABA, and waxes, surfactants, occlusives, hygroscopic agents, emulsifiers, emollients, lipid-free cleansers, antioxidants and lipophilic agents, may be added to the topical formulations of the disclosure if desired. A topical formulation of the media and/or cells of the disclosure may be designed to be left on a tissue and not washed shortly after application. Alternatively, the topical formulation may be designed to be rinsed off within a given amount of time after application.

In one embodiment, the treatment of one or more immunological diseases is performed by administration of the hypoxic/carbon monoxide fibroblast-treated conditioned media and/or the prepared fibroblasts directly to its site(s) of therapeutic activity, which in the case of many immune diseases is in the lymph nodes. For example, the therapeutic agent may be injected directly into the lymph nodes. Preferred lymph nodes for intranodal injections of inhibitors of T cell-dependent activation are the major lymph nodes located in the regions of the groin, the underarm and the neck. In another embodiment, the therapeutic agent is administered distal to the site of its therapeutic activity.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physician's Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

In specific embodiments, a medical condition treated with prepared fibroblasts and/or conditioned media therefrom and/or fibroblast-derived products include oral conditions related to autoimmune diseases, degenerative diseases, inflammatory diseases, and fibrotic diseases. Examples of autoimmune diseases are Graves' disease, lupus, type I diabetes, multiple sclerosis, rheumatoid arthritis, and so on. Examples of degenerative diseases are degenerative disc disease, Alzheimer's disease, cancer, Charcot-Marie-Tooth disease, type II diabetes, heart disease, muscular dystrophy, Parkinson's disease, Huntington's disease, macular degeneration, spinal muscular atrophy, Inflammatory bowel disease, osteoporosis, osteoarthritis, Tay-Sachs disease, primary pulmonary hypertension, and so on. Examples of inflammatory diseases (that can be chronic or acute) include asthma, tuberculosis, ulcerative colitis, chronic peptic ulcer, periodontitis, Crohn's disease, sinusitis, and so on. Examples of fibrotic disease include Pulmonary fibrosis, radiation-induced lung injury, cirrhosis, atrial fibrosis, endomyocardial fibrosis, glial scar, keloid, myelofibrosis, scleroderma, and so on.

It is known that gum disease is associated with inflammation. In some cases, the inflammation is chronic and is associated with deterioration of gingival tissue. Gum disease does not only occur from poor hygiene and bacterial infection/dysbiosis, but may also occur as the results of radiation therapy or other deterioration diseases. It known that gum disease, in some instances, is associated with enhanced levels of proteolytic enzymes such as MMPs, elastases, and other proteinases, which contribute to tissue destruction. Accordingly, in some embodiments of the disclosure, fibroblasts are treated to enhance expression of inhibitors of proteolytic enzymes. Since it is known that under some conditions fibroblasts are capable of producing MMP activity, in some embodiments of the disclosure, ability of fibroblasts to produce pathological enzymes is deleted by use of gene editing for permanent deletion, or use of RNA interference, ribozymes or antisense oligonucleotides for transient suppression. It is known that cellular administration into the inflamed gingival environment may cause cells which are normally regenerative to take on fibrotic, or inflammatory features. Accordingly, in some embodiments of the disclosure, fibroblasts are administered with various anti-inflammatory agents. In one embodiment, fibroblasts are administered together with agents selected from a group comprising of: cycloheximide, auranofin, sodium aurothiomalate, Leukotriene B4, interleukin-4, interleukin-13, polymyxin B, bile acids, interleukin-6, lactulose, oxpentifylline, mometasone, glucocorticoids, colchicine, chloroquine, FK-506, berberine, resveratrol, pterostilbene, vitamin A, vitamin C, cyclosporine, phosphodiesterase inhibitors such as vinpocetine, milrinone, CI-930, rolipram, nitroquazone, zaprinast, synthetic lipid A, melatonin, amrinone, N-acetylcysteine, dithiocarbamates and metal chelators, exosurf synthetic surfactant, dehydroepiandrosterone, delta-tetrahydrocannabinol, phosphatidylserine, TCV-309, a PAF antagonist, thalidomide, a cytochrome p450 inhibitor, cytochalasin D, ketamine, TGF-beta, interleukin-10, pentoxifylline, BRL 61,063, a calcium antagonist, curcumin, kappa-selective opioid agoinst U50,488H (trans-3,4-dichloro-N-methyl-N-[7-(1-pyrrolidinyl)cyclohexyl]benzene-acetamide methanesulfonate), alendronate, tetrandrine, sulfasalazine, epinephrine, BMS-182123, adenosine, E3330, nicotine, IVIG, cardiotrophin-1, KB-R7785, CGRP, ligustrazine, dexanabinol, iloprost, activated protein C, a growth hormone, spermine, FR-167653, gm-6001, estradiol, aspirin, or amiodarone.

For the practice of some embodiments of the disclosure, fibroblasts are chosen for anti-inflammatory, tissue regenerating, and/or antimicrobial properties. In some embodiments, fibroblasts are administered with inhibitors of TNF-alpha prior to administration into the oral cavity in order to allow the fibroblasts to maintain regenerative properties. Numerous inhibitors of TNF-alpha are known and some are provided for reference in the following. In some embodiments, inhibitors of the effects of TNF-alpha production are administered either systemically and/or locally to suppress inflammation and allow for enhancement of therapeutic effects of cells and/or regenerative factors administered. Some examples of agents which inhibit activities of TNF-alpha include; ibuprofen and indomethacin, Nedocromil sodium and cromolyn (sodium cromoglycate), spleen derived factors, pentoxifylline, the 30 kDa TNF-alpha inhibitor, NG-methyl-L-arginine, antibodies directed against the core/lipid A, dexamethasone, chlorpromazine, activated alpha 2 macroglobulin, serum amyloid A protein, neutrophil derived proteolytic enzymes, phentolamine and propranolol, leukotriene inhibitors, nordihydroguaiaretic acid, genistein, butylated hydroxyanisole, CNI-1493, quercetin, gabexate mesylate, SM-12502, monoclonal nonspecific suppressor factor (MNSF), pyrrolidine dithiocarbamate (PDTC), and aprotinin.

It is known that under certain conditions fibroblasts are capable of producing interleukin-1 and/or other inflammatory cytokines. The disclosure encompasses that fibroblasts may be treated by gene editing of IL-1 and/or other inflammatory mediators in order to prevent expression of inflammatory cytokines by fibroblasts after administration into the oral cavity. In some embodiments of the disclosure, TNF-alpha and inflammatory mediators are suppressed in the oral cavity, however, fibroblasts may be pretreated with TNF-alpha in a manner to induce expression of growth factors and/or proliferation such as described in this following publication and incorporated by reference.

Compositions of the present disclosure may be obtained from isolated fibroblast cells or a population thereof capable of proliferating and differentiating into ectoderm, mesoderm, or endoderm. In some embodiments, the fibroblasts possess the ability to differentiate to osteogenic, chondrogenic, and adipogenic lineage cells. In some embodiments, the enriched population of fibroblast cells includes cells that are about 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 micrometers in size. In some embodiments, the fibroblast cells are expanded in culture using one or more cytokines, chemokines and/or growth factors prior to administration to an individual in need thereof. The agent capable of inducing fibroblast expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family. The agent capable of inducing fibroblast differentiation can be selected from HGF, BDNF, VEGF, FGF1, FGF2, FGF4, and FGF20.

In some embodiments, fibroblasts of the present disclosure are adherent to plastic. In some embodiments, the fibroblasts express CD73, CD90, and/or CD105. In some embodiments, the fibroblasts are CD14, CD34, CD45, and/or HLA-DR negative.

In some embodiments, the fibroblast cells express proteins characteristic of normal fibroblasts including the fibroblast-specific marker, CD90 (Thy-1), a 35 kDa cell-surface glycoprotein, and the extracellular matrix protein, collagen. In some embodiments, an isolated fibroblast cell expresses at least one of Oct-4, Nanog, Sox-2, KLF4, c-Myc, Rex-1, GDF-3, LIF receptor, CD105, CD117, CD344, or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media.

In some embodiments, the method optionally includes the step of depleting cells expressing stem cell surface markers or MHC proteins from the cell population, thereby isolating a population of stem cells. In some embodiments, the cells to be depleted express MHC class I, CD66b, glycophorin a, or glycophorin b. In some embodiments, fibroblast cells are isolated and expanded and possess one or more markers selected from a group consisting of CD10, CD13, CD44, CD73, CD90, CD141, PDGFr-alpha, HLA-A, HLA-B, HLA-C, or a combination thereof. In some embodiments, the fibroblast cells do not produce one or more of CD31, CD34, CD45, CD117, CD141, HLA-DR, HLA-DP, HLA-DQ, or a combination thereof. In further embodiments, the fibroblast regenerative cell has enhanced expression of GDF-11 as compared to a control. In still further embodiments, the fibroblast cells express CD73, which is indicative of fibroblast cells having regenerative activity.

In some embodiments, fibroblasts of the present disclosure express telomerase, Nanog, Sox2, β-III-Tubulin, NF-M, MAP2, APP, GLUT, NCAM, NeuroD, Nurrl, GFAP, NG2, Oligl, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5, TrkA, BMP2, BMP4, FGF2, FGF4, PDGF, PGF, TGFα, TGFβ, and/or VEGF.

In some embodiments, fibroblast regenerative cells comprise fibroblast side population cells isolated based on expression of the multidrug resistance transport protein (ABCG2) or the ability to efflux intracellular dyes such as rhodamine-123 and or Hoechst 33342. Without being bound to theory, cells possessing this property express stem-like genes and are known for enhanced regenerative ability compared to cells which do not possess this efflux mechanism. Fibroblast side population cells are derived from tissues including pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, or a combination thereof.

In some embodiments, fibroblast populations are utilized in an autologous manner. In another embodiment, allogeneic fibroblast populations are utilized for the practice of the disclosure. In another embodiment, xenogeneic fibroblast populations are used. In some cases, the fibroblasts are selected for expression of CD73. In some embodiments, fibroblasts are selected from a population of fibroblasts that are extracted from a tissue. Various tissue sources of fibroblast populations may be used for the practice of the disclosure, these include one or more of skin, placenta, adipose, bone marrow, peripheral blood, omentum, and Wharton's jelly, foreskin, umbilical cord, amniotic fluid, umbilical cord blood, ear lobe skin, embryonic fibroblasts, plastic surgery-related by-product, and nail matrix. In certain embodiments, fibroblast populations are obtained from a punch biopsy and/or cultured in a hypoxic environment during dissociation of tissue to yield a cellular population, placental tissue dissected so as to obtain fibroblast populations associated with fetal-maternal interphase, or collection of fibroblast populations that are in contact with placental blood vessels. In some embodiments, adipose-derived fibroblast populations are collected from stromal vascular fraction of adipose tissue, the stromal vascular fraction of which may contain less than 5% contamination by adipocytes. Bone marrow-derived fibroblast populations may be obtained from bone marrow closer to the outside of the marrow compartment, and in proximity to the bone itself, or selected from the hypoxic area of the bone marrow. In some embodiments, fibroblast populations are substantially free of hematopoietic stem cells. In some embodiments, fibroblast populations contain less than 5 percent of CD34, CD33, and CD45 cells. In some embodiments, peripheral blood derived fibroblast populations are purified by using a density gradient and selecting for mononuclear cells, wherein said mononuclear cells are separated into adherent and non-adherent cells, wherein said adherent cells may be allowed to proliferate in tissue culture for a period sufficient to allow for fibroblast outgrowth over adherent monocytic lineage cells, and wherein said adherent peripheral blood-derived mononuclear cells are cultured for a period of 3 day to 3 years or for a period of longer than three days, and cultured in RPMI-1640-, OPTI-MEM- or DMEM-based media with fetal calf serum or platelet lysate. Peripheral blood-derived fibroblast populations may be derived from peripheral blood of a patient treated with an agent capable of mobilizing fibroblast populations into systemic circulation, wherein said agent is selected from a group comprising of G-CSF, GM-CSF, M-CSF, FLT-3L, and Mozobil.

In some embodiments, the fibroblast population is treated under certain conditions to enhance therapeutic efficacy for gingival regeneration before administration. One condition capable of enhancing regenerative activity is culture in the presence of hypoxia, wherein said hypoxia is sufficient to induce nuclear translocation of hypoxia inducible factor (HIF)-1 in at least 80% of cultured fibroblasts and/or said hypoxia is sufficient to induce a 50% or greater induction of VEGF, HGF-1, BMP-2, LL-37, PD-L1, HLA-G, IL-10, and/or TGF-beta in at least 80% of cultured fibroblasts. Another condition capable of enhancing regenerative activity is culturing in the presence of an inflammatory stimuli comprising of a toll like receptor (TLR) agonist, wherein said TLR is TLR-1, TLR-2, TLR-3, TLR-4, TLR-5, TLR-6, TLR-7, TLR-8, or TLR-9, wherein said activator of TLR-1 is Pam3CSK4, said activator of TLR-2 is HKLM, said activator of TLR-3 is Poly:IC, and said activator of TLR-4 is comprised of β-defensin 2, small molecular weight hyaluronic acid, fibronectin EDA, snapin, or tenascin C, said activator of TLR-5 is flagellin, said activator of TLR-6 is FSL-1, said activator of TLR-7 is imiquimod, said activator of TLR-8 is ssRNA40/LyoVec, and said activator of TLR-9 is comprised of a CpG oligonucleotide, ODN2006, or Agatolimod.

The disclosure also concerns a device used for dental or oral implantation. In some embodiments, the device comprises a dental implant comprising an abutment portion for connecting to a tooth crown and a hollow base portion defining a cavity therein, said portion may be formed integrally with said hollow base portion. In some embodiments, a bio-supportive or biodegradable scaffold is carried by said hollow base portion of the dental implant. In some embodiments, said scaffold is impregnated with a fibroblast population, growth factor and/or bone graft material. In some embodiments, the fibroblast population is on the inside and/or outside and/or surface of the device. In other embodiments, the fibroblast population is affixing and/or coating the device. In some embodiments, the device is an implant.

In some embodiments, the fibroblasts produce more than 2 fold their basal production of VEGF, EGF, IGF, PDGF, FGF-1, FGF-2, FGF-5, HGF, angiopoietin, or PGE-2 when cultured under conditions of 1% oxygen for 12 hours. In some embodiments, said fibroblast lacks expression of HLA-II, CD14, CD34, or CD45. The disclosure encompasses a scaffold including a flexible mesh impregnated with and carrying said regenerative stem cells, said growth factor, or said bone graft material, wherein said flexible mesh is made of one of natural hydrogel, synthetic hydrogel and polymer.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of preventing and/or treating one or more oral medical conditions and/or one or more oral injuries and/or one or more conditions from treatments thereof, comprising the step of administering a therapeutically effective amount of a fibroblast population and/or derivatives thereof to the oral cavity of an individual in need thereof.

2. The method of claim 1, wherein one or more medical conditions and/or one or more oral injuries and/or one or more conditions from treatments thereof is of soft tissue of the oral cavity of the individual.

3. The method of claim 1 or 2, wherein the soft tissue is the lips, tongue, cheek, gingiva, mucosa, uvula, soft palate, frenulum, or a combination thereof.

4. The method of any one of claims 1-3, wherein one or more medical conditions and/or one or more oral injuries and/or one or more conditions from treatments thereof is of hard tissue of the oral cavity of the individual.

5. The method of claim 4, wherein the hard tissue is the bone, hard palate, or tooth or a combination thereof.

6. The method of any one of claims 1-5, wherein the oral medical condition is tooth loss, tooth decay, tooth impaction, gum disease, periodontitis, abscess, mouth ulcer, cold sore, rash, inflammation, leukoplakia, halitosis, infection, microbiome dysbiosis, bacterial microfilm, cancer, or a combination thereof.

7. The method of any one of claims 1-6, wherein the injury is blunt force, puncture, fracture, luxation, soft tissue injury, burn, foreign body intrusion, gum trauma, dental and/or orthodontic mismanagement, or a combination thereof.

8. The method of any one of claims 1-6, wherein the oral medical condition and/or oral injury and/or conditions from treatment comprises surgery, radiation, drug therapy, chemotherapy, or immunotherapy.

9. The method of any of claims 1-8, further comprising administering to the individual one or more therapies for the medical condition and/or oral injury and/or conditions from treatment thereof.

10. The method of any of claims 1-9, wherein the one or more oral medical conditions and/or one or more oral injuries and/or one or more conditions from treatment thereof is associated with enhanced production of one or more proteases and/or reduced production of one or more protease inhibitors in the soft tissue and/or hard tissue.

11. The method of claim 10, wherein said one or more protease inhibitors are one or more tissue inhibitors of metalloproteases.

12. The method of any one of claims 1-11, wherein said fibroblast population is allogenic, xenogeneic, or autologous with respect to the individual.

13. The method of any one of claims 1-12, wherein said fibroblast population comprises CD73-positive fibroblasts.

14. The method of any one of claims 1-13, wherein the method comprises a step of selection of CD73-positive fibroblasts.

15. The method of claim 14, wherein said selection of CD73-positive fibroblasts is performed on a population of fibroblasts extracted from a tissue.

16. The method of claim 15, wherein said tissue comprises at least one of skin, placenta, adipose, bone marrow, peripheral blood, omentum, Wharton's jelly, foreskin, umbilical cord, amniotic fluid, umbilical cord blood, embryonic fibroblasts, plastic surgery-related by-product, nail matrix, or a combination thereof.

17. The method of claim 16, wherein the skin tissue is obtained from a punch biopsy.

18. The method of claim 16 or 17, wherein a resulting skin fibroblast population is cultured in a hypoxic environment during dissociation of tissue to yield a cellular population.

19. The method of claim 16, wherein said placental tissue is from the fetal-maternal interphase.

20. The method of claim 16 or 19, wherein said placental tissue is dissected from the fetal-maternal interphase to obtain the fibroblast population.

21. The method of claim 16, 19, or 20, wherein said fibroblast population that is collected is in contact with placental blood vessels.

22. The method of claim 16, wherein the adipose is from a stromal vascular fraction of adipose tissue.

23. The method of claim 22, wherein said stromal vascular fraction of adipose tissue comprises less than 5% contamination by adipocytes.

24. The method of claim 16, wherein the bone marrow is obtained from bone marrow adjacent to the bone itself, but not further than 1 centimeter away from the bone, and in proximity to the bone itself.

25. The method of claim 16, wherein the bone marrow is selected from the hypoxic area of the bone marrow.

26. The method of claim 16 or 25, wherein the resulting CD73-positive bone marrow fibroblast population is substantially free of hematopoietic stem cells.

27. The method of claim 16, 25, or 26, wherein the resulting CD73-positive bone marrow fibroblast population comprises less than 5% CD34 cells, and/or less than 5% CD33 cells, and/or less than 5% CD45 cells.

28. The method of claim 16, wherein fibroblasts from the peripheral blood are purified using a density gradient and selecting for mononuclear cells.

29. The method of claim 28, wherein said mononuclear cells are separated into adherent and non-adherent cells.

30. The method of claim 29, wherein said adherent cells are allowed to proliferate in tissue culture for a period sufficient to allow for fibroblast population outgrowth over adherent monocytic lineage cells.

31. The method of claim 30, wherein said adherent cells are cultured for a period of 3 days to 3 years.

32. The method of claim 30, wherein said adherent cells are cultured for a time period of longer than 3 days.

33. The method of any one of claims 30-32, wherein said adherent cells are cultured in media based on one of RPMI-1640, OPTI-MEM, and/or DMEM with fetal calf serum and/or platelet lysate.

34. The method of claim 16, wherein the peripheral blood is peripheral blood of an individual treated with one or more agents capable of mobilizing fibroblast populations into systemic circulation.

35. The method of claim 34, wherein said one or more agents capable of mobilizing fibroblast populations into systemic circulation comprises at least one of G-CSF, GM-CSF, M-CSF, FLT-3L, and/or Mozobil.

36. The method of any one of claims 1-35, wherein said fibroblast population is treated under one or more conditions to enhance therapeutic efficacy for gingival regeneration prior to and/or during and/or following administration.

37. The method of claim 36, wherein said one or more conditions capable of enhancing regenerative activity comprises culture in the presence of hypoxia, carbon monoxide, or a combination thereof.

38. The method of claim 37, wherein the carbon monoxide is provided to the fibroblasts before the hypoxia.

39. The method of claim 37, wherein the carbon monoxide is provided to the fibroblasts subsequent to the hypoxia.

40. The method of claim 37, wherein the carbon monoxide is provided to the fibroblasts at the same time as the hypoxia.

41. The method of claim 37, wherein said hypoxia is sufficient to induce nuclear translocation of hypoxia inducible factor (HIF-1) in at least 80% of cultured fibroblasts.

42. The method of claim 37, wherein said hypoxia is sufficient to induce a 50% or greater induction of any one or more of VEGF, HGF-1, BMP-2, LL-37, PD-L1, HLA-G, IL-10, and/or TFG-beta in at least 80% of cultured fibroblasts.

43. The method of claim 36, wherein said condition capable of enhancing regenerative activity comprises culture in the presence of at least one inflammatory stimulus.

44. The method of claim 43, wherein said inflammatory stimulus comprises a Toll-like receptor (TLR) agonist.

45. The method of claim 44, wherein said Toll-like receptor is TLR-1.

46. The method of claim 45, wherein said agonist of TLR-1 is Pam3CSK4.

47. The method of claim 44, wherein said Toll-like receptor is TLR-2.

48. The method of claim 47, wherein said agonist of TLR-2 is HKLM.

49. The method of claim 44, wherein said toll like receptor is TLR-3.

50. The method of claim 49, wherein said agonist of TLR-3 is Poly:IC.

51. The method of claim 44, wherein said Toll-like receptor is TLR-4.

52. The method of claim 51, wherein said agonist of TLR-4 is any one of LPS, Buprenorphine, Carbamazepine, Fentanyl, Levorphanol, Methadone, Cocaine, Morphine, Oxcarbazepine, Oxycodone, Pethidine, Glucuronoxylomannan from Cryptococcus, Morphine-3-glucuronide, lipoteichoic acid, β-defensin 2, a small molecular weight hyaluronic acid, fibronectin EDA, snapin, tenascin C, or a combination thereof.

53. The method of claim 44, wherein said Toll-like receptor is TLR-5.

54. The method of claim 53, wherein said agonist of TLR-5 is flagellin.

55. The method of claim 44, wherein said Toll-like receptor is TLR-6.

56. The method of claim 55, wherein said agonist of TLR-6 is FSL-1.

57. The method of claim 44, wherein said Toll-like receptor is TLR-7.

58. The method of claim 57, wherein said agonist of TLR-7 is imiquimod.

59. The method of claim 44, wherein said Toll-like receptor is TLR-8.

60. The method of claim 59, wherein said agonist of TLR-8 is ssRNA40/LyoVec.

61. The method of claim 44, wherein said Toll-like receptor is TLR-9.

62. The method of claim 61, wherein said agonist of TLR-9 is any one of a CpG oligonucleotide, ODN2006, and/or Agatolimod.

63. A device for oral use comprising a fibroblast population.

64. The device of claim 63 further comprising one or more growth factors, bone graft material, or a combination thereof.

65. The device of claim 63 or 64, wherein the fibroblast population is on an interior and/or exterior of the device.

66. The device of any one of claims 63-65, wherein the fibroblast population is on a surface of the device.

67. The device of any one of claims 63-66, wherein the fibroblast population is affixed to and/or a coating on the device.

68. The device of any one of claims 63-67, wherein the device is an implant.

69. The device of any one of claims 63-68, wherein the device is administered to an individual, and said fibroblast population is allogenic, xenogeneic, or autologous with respect to the individual.

70. The device of any one of claims 63-69, wherein said fibroblast population comprises CD73-positive fibroblasts.

71. The device of any one of claims 63-70, wherein said fibroblasts are from skin, placenta, adipose, bone marrow, peripheral blood, omentum, and Wharton's jelly, foreskin, umbilical cord, amniotic fluid, umbilical cord blood, embryonic fibroblasts, plastic surgery-related by-product, nail matrix, or a combination thereof.

72. The device of claim 71, wherein said skin is from a punch biopsy.

73. The device of claim 71 or 72, wherein said fibroblasts from skin have been exposed to hypoxia.

74. The device of claim 71, wherein said placenta is from the fetal-maternal interphase.

75. The device of claim 71, wherein said adipose is from the stromal vascular fraction of adipose tissue.

76. The device of claim 75, wherein said stromal vascular fraction of adipose tissue contains less than 5% contamination by adipocytes.

77. The device of claim 71, wherein said bone marrow is from bone marrow closer to the outside of the marrow compartment, and in proximity to the bone itself.

78. The device of claim 77, wherein said bone marrow is selected from the hypoxic area of the bone marrow.

79. The device of claim 77 or 78, wherein fibroblsats from said bone marrow are substantially free of hematopoietic stem cells.

80. The device of any one of claim 71, 77, 787, or 79, wherein fibroblasts from said bone marrow comprises less than 5% CD34 cells, and/or less than 5% CD33 cells, and/or less than 5% CD45 cells.

81. The device of claim 71, wherein said peripheral blood is from peripheral blood of an individual treated with one or more agents capable of mobilizing fibroblast populations into systemic circulation.

82. The device of claim 81, wherein said one or more agents capable of mobilizing fibroblast populations into systemic circulation comprises at least one of G-CSF, GM-CSF, M-CSF, FLT-3L, and/or Mozobil.

83. The device of any one of claims 63-82, wherein said fibroblast population were previously treated under conditions to enhance therapeutic efficacy for gingival regeneration prior to and/or during administration.

84. The device of claim 83, wherein said condition capable of enhancing regenerative activity is culture in the presence of hypoxia, carbon monoxide, or a combination thereof.

85. The device of claim 84, wherein the carbon monoxide is provided to the fibroblasts subsequent to the hypoxia.

86. The device of claim 84, wherein the carbon monoxide is provided to the fibroblasts at the same time as the hypoxia.

87. The device of claim 84, wherein said hypoxia is sufficient to induce nuclear translocation of hypoxia inducible factor (HIF-1) in at least 80% of cultured fibroblasts.

88. The device of claim 84 or 87, wherein said hypoxia is sufficient to induce a 50% or greater induction of any one of VEGF, HGF-1, BMP-2, LL-37, PD-L1, HLA-G, IL-10, and/or TFG-beta in at least 80% of cultured fibroblasts.

89. The device of any one of claims 63-88, wherein said fibroblast population produces more than 2 fold its basal production of any one or more of BEGF, EGF, IGF, PDGF, FGF-1, FGF-2, FGF-5, HGF, angiopoietin, and PGE-2 when cultured under conditions of 1% oxygen for 12 hours.

90. The device of any one of claims 63-89, wherein said fibroblast population lacks expression of any one or more of HLA-II, CD14, CD34, and CD45.

91. The device of any one of claims 63-90, wherein the device comprises a scaffold comprising a flexible mesh impregnated with and carrying the fibroblast population, growth factors, and/or bone graft material.

92. The device of claim 91, wherein said flexible mesh is comprised of one or more of natural hydrogel, synthetic hydrogel, or polymer.

93. A method of preventing and/or treating one or more oral medical conditions and/or one or more oral injuries and/or one or more conditions from treatments thereof, comprising the step of administering a therapeutically effective amount of a stem cell population and/or derivatives thereof to the oral cavity of an individual in need thereof.
